(19) United States
(12) Patent Application Publication
(10) Pub. No.: US 2015/0230420 A1
(43) Pub. Date: Aug. 20, 2015

(54) PHOTOBIOREACTOR FOR CULTIVATING PHOTOTROPHIC ORGANISMS

(71) Applicant: COMITEN GBR, Bad Honnef (DE)

(72) Inventor: Jan Horst Moddemann, Bad Honnef (DE)

(21) Appl. No.: 14/421,613

(22) PCT Filed: Jul. 31, 2013
(86) PCT No.: PCT/EP2013/066108

§ 371 Date: Feb. 13, 2015

(30) Foreign Application Priority Data
Aug. 14, 2012 (DE) 10 2012 214 493.5

(51) Int. Cl. A01G 33/00; C12M 3/00; C12M 1/00

(52) U.S. Cl. CPC A01G 33/00; C12M 21/02; C12M 23/48

(57) ABSTRACT

The invention relates to a photobioreactor for the cultivation of phototrophic organisms, particularly algae, an arrangement of a supporting member in a photobioreactor, as well as to a biogas unit equipped with such a photobioreactor. According to the invention, a transparent pipe system for the flow-through of a culture suspension, in particular algae substrate, is provided. In this case, the transparent pipe system is configured in the form of levels in order to enable a particularly efficient cultivation over several levels.

The invention relates to a photobioreactor according to the preamble of claim 1, an arrangement of a supporting member in a photobioreactor according to the co-ordinated claim as well as to a biogas unit equipped with such a photobioreactor.

Due to their property of being a rich, high-quality biomass, phototrophic organisms such as algae can advantageously be used for the production of biogas. In the process, the phototrophic organisms are cultivated in special photobioreactors and then fermented to biogas under as optimal conditions as possible in a fermenter—also referred to as a bioreactor.

Open and closed production systems are known for the cultivation of the phototrophic organisms. In the case of open production systems, such as an open basin, cultivation takes place outdoors, and the light required for the growth of the phototrophic organisms is substantially provided by the available sunlight. In this case, however, there is a danger of contamination in the cultivation of the organisms, particularly due to environmental influences such as wind and foreign substances, which may be introduced from above due to the open arrangement. In the end, they may also cause a mutation of the cultivated organisms into unknown or harmful algae, for example, and thus also contaminate other parts of the unit. The targeted temperature regulation of the production system, which forms an essential factor in the cultivation and is expediently between 16-22° c for algae, for example, is troublesome to realize.

A closed production system in which both sunlight as well as artificial light is used for the cultivation of phototrophic microorganisms is known from document DE 41 34 813 A1. The device comprises channels in the shape of meandering lines through which a culture medium flows. The channels are disposed between plane-parallel plates and, suspended on a frame, can be pivoted for an improved incidence of light in one embodiment.

Furthermore, a coupling of a photobioreactor with a biogas unit is known (DE 10 2010 001 907 A1), in which a photobioreactor with structural pipes laid in a meandering shape is provided. The structural pipes arranged with an inclination angle have a special surface pattern in order to prevent the formation of a biofilm.

The complex structure, which makes an industrial-scale realization for an economic cultivation of phototrophic organisms difficult, is problematic in such closed production systems. The photobioreactors of the type mentioned in the introduction are primarily designed for a limited production volume. In the case of industrial-scale photobioreactors or biogas units it is also necessary to design them in a maintenance-friendly manner in order to increase the system availability and ensure optimum operation.

Furthermore, plant or animal residual matter and organic waste are increasingly used as a substrate for the cultivation of phototrophic organisms and for producing biogas. Large quantities of these are produced, in particular, in densely populated settlement areas, where, however, it is generally impossible to allow for large-scale photobioreactors or biogas units in urban planning. Generally, photobioreactors or biogas units are provided in remote areas. The necessary transport of substrate material in turn affects the cost-effectiveness of a photobioreactor or of a biogas unit.

Unless otherwise stated below, the above-mentioned features can be combined in any manner, individually or in any combination, with the subject matter of the invention described below.

It is an object of the present invention to further develop a photobioreactor or a biogas unit.

The object of the invention is accomplished by a photobioreactor having the features of claim 1 and by an article having the features of the co-ordinated claim. Advantageous embodiments are apparent from the dependent claims.

Thus, according to the proposal, a transparent pipe system for the flow-through of a culture suspension is provided, preferably in such a way that the pipes for the growth of the phototrophic organisms are oriented substantially horizontally. A culture suspension within the sense of the invention comprises a nutrient solution for the cultivation of phototrophic organisms. In the case of a cultivation of algae, the culture suspension in particular constitutes an algae substrate. Moreover, the transparent pipe system is configured in levels, in particular in such a way that at least two levels, preferably more than four levels, are created. The configuration of the pipe system in levels thus corresponds to a substantially horizontal extent of the pipes on several levels. Preferably, the spacing of the pipes or pipe sections within a level is considerably smaller than the spacing between the levels. A space-saving arrangement of the pipes or pipe sections on the levels is thus ensured. The configuration of the transparent pipe system in levels consequently enables an efficient use of surface area. The requirements as regards the surface area of a photobioreactor or of a biogas unit equipped with such a photobioreactor can be reduced in this manner. At the same time, a large pipe volume for the flow-through of a culture suspension, such as algae substrate, is provided in an efficient manner, which is sufficiently exposed to light for an optimum photosynthesis due to the configuration in levels. Undesirable shading of the pipe system for the flow-through of a culture suspension, as it is common e.g. in the case of a micro algae substrate, is thus avoided. Consequently, the efficiency of a photobioreactor or of a biogas unit on the whole is improved.

In order to provide protection for the transparent pipe system against the influence of the weather and a thermal insulation, an outer shell, which is light-transmissive in some areas, is provided in one embodiment. The upper part of the outer shell, in particular the roof, is preferably configured in a light-transmissive manner in order to ensure an incidence of daylight that is as optimal as possible.

In a preferred embodiment, the roof is configured for variable light transmittance. Preferably, a double-walled structure in the form of a cover for the flow-through of a gas is provided on the roof. The double-walled cover is preferably configured as a film, for example consisting of plastic, such as polyethylene or graphene, in particular with a very good light transmittance. Advantageously, the graphene is present as a layer, which is configured in such a way, in particular, that the light transmittance can be varied by applying a voltage, for example by magnetization of metal particles, such as fine metal chips, in a liquid. Accordingly, a magnetizable liquid can be made to flow through the double-walled structure. Preferably, the light transmittance is adjustable in such a way that a reflectance of 50% to less than 3% can be obtained. The arrangement of the film as a cover is preferably realized in such a way that the film can be inflated by an overpressure in the photobioreactor and thus forms a stable roof. Accordingly, the double-walled cover can be sealed on the outer shell and the wall of the photobioreactor by means of sealing connecting members, such as clamping rings. Gas can thus flow through the double-walled cover in order to attain a variable light transmittance of the roof. The gas that can be conducted in the double-walled cover can advantageously be selected from the group including methane, propane, butane or ethane. The gas is made to flow through in such a way that the light transmittance of the roof and thus the lighting of the pipe system on the inside is adapted in a targeted manner, depending on the external ambient conditions. In the summer, or in the case of a strong incidence of light, the light transmittance can thus be reduced, particularly with regard to damaging UV rays, in order to adapt the incidence of light for an optimum growth of phototrophic organisms. In principle, it is also possible to provide a double-walled cover made from plates consisting of glass or plastic, for example.

In a preferred embodiment, the roof comprises a roof structure, preferably in the form of a framework structure. If necessary, the double-walled cover can then be arranged over the roof structure. Furthermore, the roof structure preferably has means for lifting and lowering the levels of the pipe system. In this case, the levels can comprise—in a manner to be explained later—retaining mechanisms for the pipes of the pipe system. It is constructionally particularly advantageous to provide cable winches on the roof structure in order to enable a flexible height adjustment of the levels or retaining mechanisms and thus of the individual pipes or pipe sections through cables. In particular, the roof structure is in that case simultaneously configured as a bridge structure in order to be able to move the cable winches horizontally via rollers. Accordingly, the spacing between the levels of the pipe system can be altered in a variable manner, so that a compact arrangement of the pipe system within the photobioreactor is possible, which can be flexibly adjusted particularly for a maintenance of the pipe system. In another embodiment, a carriage, crane or the like is provided on the roof structure, in particular on the bridge structure, which can then also be moved via rollers in order to ensure convenient and safe maintenance.

The length of the transparent pipe system for the flow-through of a culture suspension, in particular an algae substrate, is preferably at least several kilometers, more preferably at least 10 km. In order to provide a circulation process, the transparent pipe system is configured in a basically closed manner in one embodiment. Accordingly, a culture suspension, such as an algae substrate, can be continuously circulated in the pipe system. In it, the phototrophic organisms can be cultivated over several days. For this purpose, the culture suspension is made to flow through the pipe system with, in particular, a correspondingly slow speed. Preferably, the speed is adjustable and is controlled depending on the desired growth rate of the phototrophic organisms. The pipes of the pipe system are preferably configured in a tube-shaped manner for an optimum flow-through. The diameter of the pipes is preferably at least 70 mm, more preferably no more than 150 mm. Preferably, plastic or glass is used as the material for the pipes. More preferably, a light plastic, particularly also including a graphene coating, is used.

The culture suspension, in particular the algae substrate, preferably comprises macroalgae and microalgae. Macroalgae, such as Avrainvillea, which enable a low level of maintenance and a high growth efficiency due to their special self-cleaning properties, are preferred. The additional microalgae, such as Spirulina, for example, contribute to an increase of the energy yield of the algae substrate. In particular, they are then added in a controlled amount only, because the solar penetration in the pipes would otherwise be limited. With the combined culture suspension as an algae substrate, the efficiency of the photobioreactor and the growth of the phototrophic organisms can be increased due to the self-cleaning effect for the pipe system.

In another embodiment, the proportion of the algae in the fermenting substrate is preferably 30 to 80%. The fermenting substrate and the culture suspension may further comprise other components, such as slurry, waste, in particular from agricultural production, as well as nutrients, such as proteins, in order to increase the methane gas production in a biogas unit.

In order to enable an improved maintenance of the transparent pipe system, the pipe system comprises separable sections in a preferred embodiment. Preferably, the pipe sections of one level are configured to be separable from each other. The option of separating sections of the pipes enables the pipe system to be maintained section by section. Individual sections can thus be inspected and repaired. Particularly preferably, the arrangement of the pipe system is configured in such a way that pipe sections can be separated without interrupting the entire pipe system. For this purpose, corresponding bypass pipes can be provided so that a maintenance of pipe sections is possible substantially without interrupting operation.

In another embodiment, one or several levels of the transparent pipe system comprise at least one retaining mechanism for the pipe system. The retaining mechanism is preferably configured in a spoke-like manner and disposed in such a way, in particular, that the retaining mechanism extends substantially starting from the inner region of the photobioreactor to the outer shell. The pipe system can then be laid on the retaining mechanism in such a way that a uniform load transfer takes place. Preferably, guides that enable a positive accommodation of the pipes are provided on the retaining mechanism. In addition, it is preferred to configure the retaining mechanism for a vertically offset and/or laterally offset arrangement of the pipes. For this purpose, the retaining mechanism comprises elevated and lowered guides. Due to the offset arrangement of the pipes, a shading of pipes that are situated side-by-side or one underneath the other can be reduced, and consequently the lighting of the pipe system as a whole can be increased. It is also advantageous to arrange the pipes in a coil shape on the retaining mechanism, in particular in a double-spiral shape.

In a preferred embodiment, the pipes of the transparent pipe system, at least in some regions, are connectable or connected to the retaining mechanism, preferably a spoke-shaped retaining mechanism, in a positive and/or frictional manner, preferably through a disengageable locking means. The positive connection can be effected via the guides in the retaining mechanism. In addition, a locking means, in particular a disengageable locking means, such as a hook-and-loop fastener, can be provided. The arrangement of the locking means is preferably configured in such a way that the contact regions between the retaining mechanism and the pipes are equipped with a corresponding locking means, for example a strap of a hook-and-loop fastener. Thus, a firm connection between the pipes of the pipe system and the retaining mechanism is created which provides for a stable cohesion of the assemblage thus produced. Consequently, the load carrying capacity is increased as a whole. In another embodiment, another fixing member is additionally provided, such as a wire, a strap or the like, for fastening the pipes to the retaining mechanism. The fixing member, such as a wire, can then be stretched in a resilient manner over the pipes in order thus to firmly press the pipes into the retaining mechanism. Accordingly, this improves the connection of the pipe system to the retaining mechanism.

In a preferred embodiment, the levels of the transparent pipe system are configured to be movable horizontally and/or vertically. For horizontally moving the level, such as when rotating the level, the levels are preferably supported in a rolling manner. The levels are preferably moved vertically via a vertical movement device, such as a motor-driven rack-and-pinion drive. The horizontal and/or vertical movability of the levels enables a flexible orientation for optimum lighting. Furthermore, the movability increases the flexibility during maintenance. It is particularly advantageous to configure the light-transmissive supporting members for horizontally moving the levels. Rails for horizontally rotating the levels can be provided on the supporting members for this purpose.

It is also possible to vertically adjust the pipe system, and if necessary the retaining mechanism of the pipe system, in particular by means of hydraulic drives. For this purpose, hydraulic cylinders can be provided on the tiers of the pipe system, which enable a vertical adjustment via spindle drives on the outer wall portion of the photobioreactor and thus change the flow rate of the culture suspension, such as an algae substrate, through the pipes. Furthermore, it is advantageous to provide on the pipe system a vibrator for introducing mechanical vibrations in order to stimulate the culture suspension flowing through. In particular, this increases the flow rate in the pipe system.

In a preferred embodiment, the levels of the pipe system are interconnected via flexible pipe sections. In particular, the flexible pipe section makes an expansion in length possible, so that a secure connection between the levels is ensured also when horizontally and/or vertically moving the levels.

A particularly compact arrangement of the levels of the pipe system in the photobioreactor is attained by the spacing between adjacent levels not being more, on average, than 100 cm, more preferably no more than 80 cm. In order to permit a constructionally particularly simple adjustment of the height of the levels, and thus of the spacings between the levels, the levels of the pipe system or the retaining mechanisms of the pipe system are preferably configured so as to be connectable to cables. Accordingly, the levels or retaining mechanisms of the pipe system can be connected to cable winches, to which the vibrators can also be attached, if necessary. Preferably, the cable winches are disposed on the roof structure or a bridge structure on the roof. The levels of the pipe system can thus be oriented and moved in a targeted manner as needed.

Furthermore, the flow rate in the pipes can advantageously be separately regulated in each level of the pipe system in the case of different rates of growth in the levels and congestions.

The arrangement of the pipes on the individual levels of the pipe system is preferably configured in such a way that the pipes extend horizontally in spiral shape. An improved flow through the pipes is achieved by the pipes being inclined in the direction of the flow. Accordingly, the retaining mechanism of the pipe system can then have a corresponding vertical offset for an inclination of the pipes. Given a diameter of the photobioreactor of 22 m, the vertical offset is preferably at least 10 cm, for example 20 cm.

Furthermore, in another embodiment, an artificial lighting is provided for the operation of the photobioreactor with artificial light. In principle, the artificial lighting can be disposed in a space-saving manner on the inner wall of the outer shell, like a wall paper with light-emitting diodes, in particular organic light-emitting diodes (OLED). It is particularly advantageous to provide lighting elements, in particular lighting pipes with light-emitting diodes (LED), next to or on the pipes of the transparent pipe system. In this way, a targeted artificial lighting can be introduced on the pipe system without causing a heat radiation that is detrimental to the growth of the phototrophic organisms. Preferably, the lighting pipes are disposed between the vertically offset pipes, preferably in the region that is shaded due to the arrangement of the pipes, such as in the triangle between three respectively adjacent pipes, in particular in the form of bundles of pipes. The lighting pipe can be situated on a separating layer, such as an aluminum layer, in particular in the form of a film, which is attached to the lighting pipe, for example. The arrangement of a lighting pipe provides for a particularly efficient lighting of pipes that are closely adjacent to each other and thus reduces the shading of the pipe system. Furthermore, the lighting pipe improves the operation of the photobioreactor on the whole in case of a reduced incidence of daylight or at night. In order to enable access to the levels of the pipe system, a vertical access element, such as a stair tower or an elevator or the like, is preferably provided. In that case, the vertical access element can have accesses or openings that enable a connection to the interior, in particular to the levels of the pipe system.

According to another aspect of the invention, at least one light-transmissive supporting member is disposed in the region of the wall of the outer shell, the supporting member being configured in a closed manner in the horizontal extent and preferably having a framework structure.

The essential point is the idea of realizing, in an advantageous manner with regard to production and assembly, an additional incidence of light by providing a horizontally continuous light-transmissive supporting member. Accordingly, the outer shell of the photobioreactor can be prepared with little effort, with the light-transmissive supporting member providing for sufficient load carrying capacity and stability of the outer shell and simultaneously improving the incidence of light from the region of the wall of the outer shell, so that lower regions of the pipe system can also be suitably lighted with daylight.

In particular, the horizontally continuous supporting member is configured in a closed manner in order to ensure a particularly high level of stability and load carrying capacity. In principle, the light-transmissive supporting members can also be configured as a post or skeleton structure. However framework structures are particularly preferred because they have a high load carrying capacity while having low weight. In particular, steel is used as the material for the supporting members.

In a preferred embodiment, the supporting members have profiled rails for coupling to the further region of the wall of the outer shell. The profiled rails are preferably provided at the lower and/or the upper end of the supporting members. In particular, the supporting members are configured in a U-shape, so that a positive coupling to the further wall region of the outer shell is created. In this way, the connection between the supporting members and wall members of the outer shell is particularly easy to realize as regards assembly. The supporting members with the profiled rails can then be placed on wall members, for example concrete members, during assembly. Then, further wall members can be placed on the supporting member. It is particularly advantageous to configure the outer shell to be substantially cylindrical. Accordingly, the light-transmissive supporting members are configured in a ring-shaped, closed manner. A particularly high level of stability of the supporting member can thus be realized even in the case of a large spatial extent of the photobioreactor. Furthermore, the light-transmissive supporting members can be clad with transparent cover members consisting of glass or plastic in order to suitably protect the interior of the photobioreactor against external influences. Also, at least two light-transmissive supporting members are preferably provided in the wall region of the outer shell. This also contributes to an improved accessibility of the individual levels or pipe sections via the vertical access elements.

In another embodiment, the outer shell comprises concrete members as wall members. In the case of a substantially cylindrical outer shell, the concrete members are preferably configured as concrete rings. Prestressed concrete is preferably used as the material, so that large supporting distances are also possible. In that case, the concrete members are, in particular, prefabricated. The concrete members contribute to the photobioreactor, as a whole, having sufficient stability due to its weight. In connection with the arrangement of light-transmissive supporting members, assembly can thus be realized in a particularly advantageous manner.

According to another aspect of the invention, a biogas unit is proposed which comprises a photobioreactor and a fermenter. With regard to the basic structure of the photobioreactor, reference may be made to the above explanations.

The essential point in the biogas unit is the idea of coupling the photobioreactor with a fermenter, with the fermenter preferably being disposed in the interior of the photobioreactor. By arranging the fermenter in such a way, an, on the whole, compact biogas unit is provided.

In an improved embodiment, a device for the temperature regulation of the photobioreactor is provided, which is substantially capable of utilizing the heat of the fermenter. This makes it possible to use the heat required for fermenting in the fermenter for the photobioreactor in an efficient manner in order to set up optimum ambient conditions for a growth of the phototrophic organisms. The carbon dioxide produced in the biogas production is expediently also used for the cultivation of the phototrophic organisms, such as algae. It is particularly advantageous to provide the fermenter centrally in the interior of the photobioreactor. Preferably, the fermenter is configured to be substantially cylindrical. The diameter of the fermenter inside the photobioreactor is in that case 10-30 m, for example.

In order to enable an efficient biogas generation, the biogas unit, in a preferred embodiment, is configured for using algae and plant residual matter, animal residual matter and/or organic waste as a substrate for the fermenter. Advantageously, fermenting residues of the fermentation are suitable as a fertilizer for the cultivation of phototrophic organisms, in particular algae. The utilization of such residual matter increases the efficiency of the biogas unit. This is particularly advantageous if the biogas unit is provided in areas with a higher population density, i.e. in particular in urban areas, so that the expenditure for providing such residual matter and/or waste is thus minimized.

In a preferred embodiment, in order to optimize the growth of the phototrophic organisms, the carbon dioxide and/or residual matter produced in the fermenter, such as fermenting residues, are fed into the pipe system of the photobioreactor as a fertilizer. Accordingly, an input device, such as a valve or the like, for supplying carbon dioxide or crude gas, such as biomethane, can be provided on the pipe system. In particular, growth of the phototrophic organisms, such as algae, is additionally promoted by the inner walls of the pipes of the photobioreactor being provided or vapor-coated with the antibacterial material graphene, a two-dimensional graphite material, in order to maintain the light transmittance of the transparent pipes. This offers protection against dirt and algae formation on the inner walls of the pipes. At the same time, gas-tightness and strength are considerably increased by the graphene layer. The process of cultivating phototrophic organisms, such as algae, as well as the harvesting process are carried out, in particular, in an automated manner.

An exemplary embodiment of the invention will be explained below based on drawings. In the drawings:

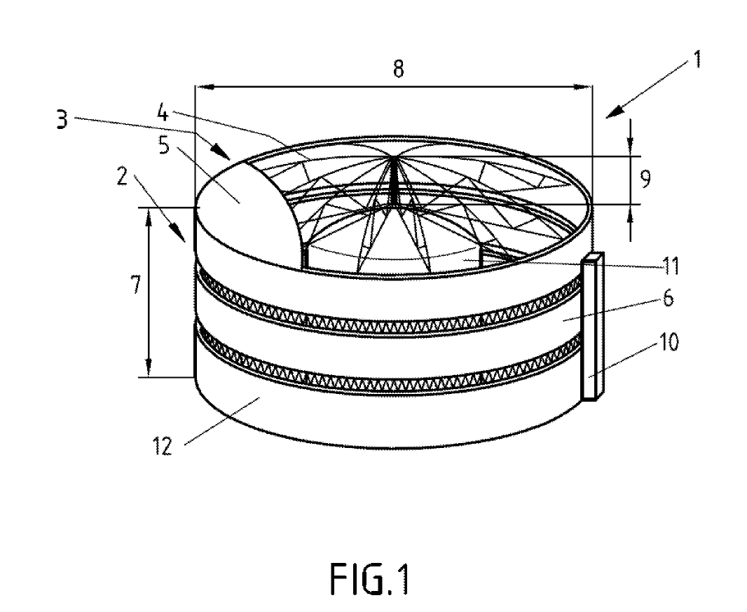

FIG. 1 shows a schematic representation of a photobioreactor or of a biogas unit,

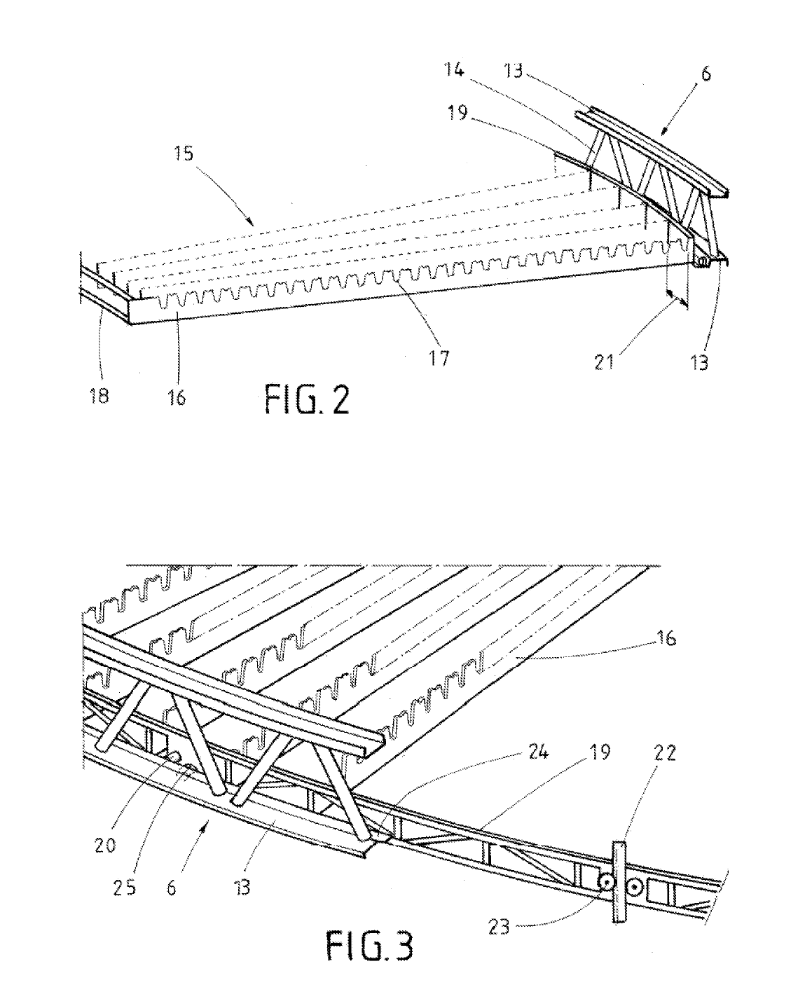

FIG. 2 shows a detailed view of a level of the pipe system,

FIG. 3 shows a detailed view of the movable level,

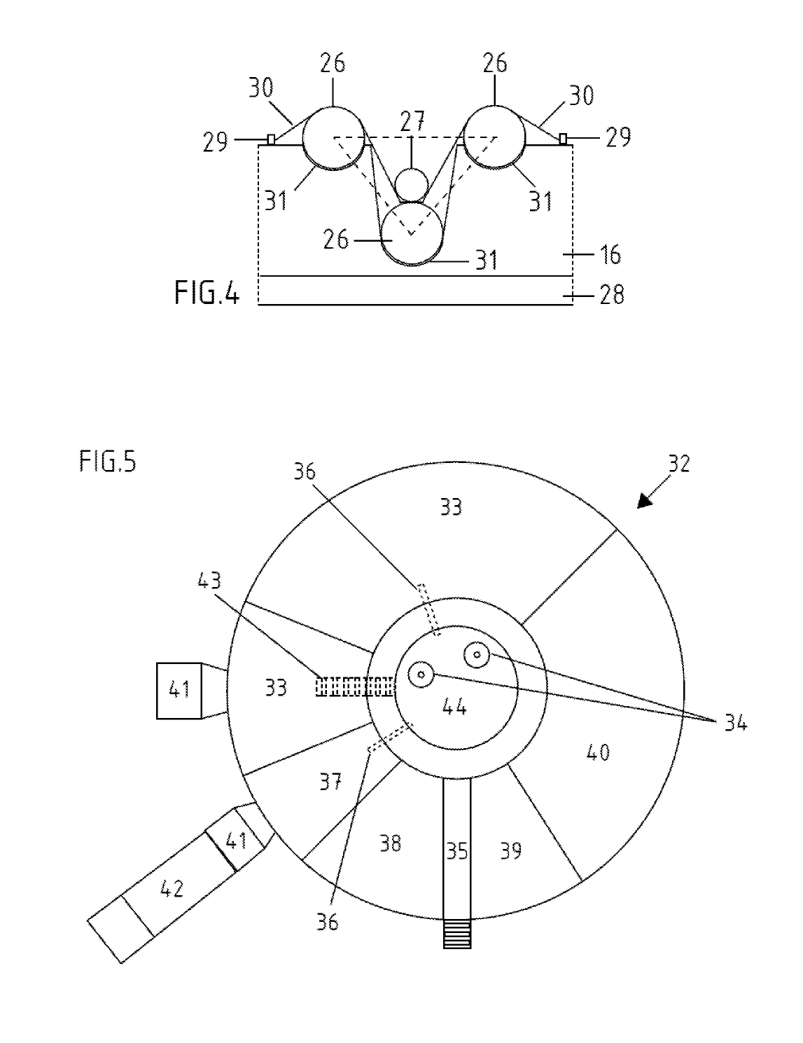

FIG. 4 shows a schematic representation of the pipes on a retaining mechanism of the pipe system, and

FIG. 5 shows a schematic representation of a basement of the photobioreactor or of the biogas unit.

The photobioreactor 1 according to FIG. 1 consists of a transparent pipe system (not shown) for the flow-through of a culture suspension, preferably with a graphene coating, particularly on the inner walls of the pipes, and an outer shell which is light-transmissive in some areas. The outer shell is preferably configured to be cylindrical and comprises a wall region 2, which extends substantially vertically. The diameter of the cylindrical shape is preferably between 30-60 m, for example 45 m. The height of the cylindrical shape, i.e. the height of the wall region 2, is preferably between 10-30 m, for example 20 m.

The upper region of the outer shell forms, in particular, a light-transmissive roof 3. The roof 3 can in this case be configured in a domed shape and thus contributes to daylight falling in an improved manner into the interior formed by the outer shell. In the center, the height 9 of the dome-shaped roof 3 protrudes over the height 7 of the wall region by at least 1 m, particularly preferably by at least 3 m. The roof 3 in particular comprises a support structure 4, which is configured as a framework structure. FIG. 1 illustrates a basic embodiment of the support structure 4, in order to apply as small additional loads as possible to the roof 3. In particular, the roof 3 is equipped with a light-transmissive cover 5 in order to protect against direct influences of the weather and prevent, in particular, the escape of gas of light, volatile methane gas.

Preferably, the cover 5 is configured for a varying light transmittance. In that case, the cover 5 can have a double-walled cover, for example consisting of polyethylene, with a graphene coating, through which a gas flows. The gas ensures that the light transmittance can be adapted in a targeted manner.

With regard to construction and assembly, it is advantageous to form the wall region 2 of the outer shell from concrete members, such as concrete rings 12, for example. In particular, prefabricated prestressed concrete can be used in that case as the concrete ring 12. In one embodiment, the wall region 2 comprises light-transmissive supporting members which, in the case of FIG. 1, are configured in a ring-shaped, closed manner as intermediate support rings 6. The intermediate support rings 6 are configured, in particular, as horizontally extending closed supporting members and, in the case of a substantially cylindrical structure of the photobioreactor 1, extend over the entire circumference of the outer shell. This not only provides for a uniform additional incidence of daylight from the wall region of the outer shell, but reduces the effort in the assembly of a photobioreactor 1 having such a structure. Furthermore, it is advantageous to provide at least two intermediate support rings 6 in the outer shell. This ensures that lower areas of the pipe system can also be expediently lighted with daylight.

In order to enable access to the interior, in particular to the transparent pipe system, a vertical access element, such as a stair tower 10 or an elevator or the like, is preferably provided. In that case, the interior, in particular the transparent pipe system, can be accessed for servicing and maintenance via openings.

According to another aspect of the invention, a fermenter 11 is provided in the interior of the photobioreactor 1, in such a way that the two are coupled to each other. The integration of the fermenter 11 in the photobioreactor 1 allows for a particularly compact and space-saving biogas unit. The fermenter 11 is preferably also configured in a cylindrical shape with a dome. The diameter of the fermenter is 10-30 m, for example, such as 20 m, for instance.

The fermenter 11 is preferably disposed centrally, so that the waste heat of the fermenter can expediently be used for the temperature regulation of the photobioreactor 1 using a temperature regulating device. Furthermore, carbon dioxide produced during biogas production and, if applicable, other residual matter, such as fermenting residues, can be used for the acceleration of growth of the phototrophic organisms, in particular algae.

Due to the integration of the fermenter 11 within the photobioreactor 1, such a biogas unit can also be advantageously provided in settlement areas, without causing considerable adverse effects from an urban planning standpoint. The compact, area-efficient structure of the photobioreactor contributes to a biogas unit of this kind avoiding urban planning limitations particularly in areas with a higher population density.

In principle, the photobioreactor 1 can also be provided without a fermenter 11 in the interior, in order to exclusively accomplish an efficient production of phototrophic organisms, such as algae, for example. In addition, it is also possible to cultivate, primarily in the summer, phototrophic organisms, such as algae, in simple concrete rings without a photobioreactor with or without a cover.

The light-transmissive roof generates a light cone in the photobioreactor, for example with a diameter of about 20 to 25 m, so that daylight penetrates down to the lower levels of the pipe system. In a photobioreactor without an integrated fermenter 11, the light can thus be guided down to the bottom of the photobioreactor. If a fermenter 11 is provided in the photobioreactor in order to obtain a biogas unit, the light cone in part ends already at the dome of the fermenter 11.

The transparent pipe system preferably consists of a flexible material, such as plastic, so that an easily handled pipe system is produced. Preferably, the pipes in the pipe system are configured in a tube-shaped manner and thus help in making it possible that the flow-through of the culture suspension, such as the algae substrate, can be carried out with a smaller energy expenditure due to the improved flow. The diameter of the pipes is preferably 50 to 150 mm, particularly preferably 80 to 120 mm. The length of the transparent pipe system is typically several kilometers, for example 10 km. Due to the transparent pipe system, the cultivation of the phototrophic organisms can take place over several days. In the case of algae, cultivation can take place, for example, within seven days, with the size of the algae substantially increasing threefold.

The flow-through of the culture suspension, such as algae substrate, in the pipe system preferably takes place with a correspondingly slow speed. The number of levels of the pipe system in a photobioreactor 1 or a biogas unit is preferably at least four, particularly preferably at least eight.

If, as shown in FIG. 1, a fermenter 11 is provided in the interior of the photobioreactor 1 in order to provide a biogas unit, the remaining space is used for the levels of the pipe system.

As is apparent from FIG. 5, storage containers for substrate and discharging devices for the cultivated organisms, such as algae, can be provided underneath the photobioreactor, in particular in a basement 32. Accordingly, transport devices for supplying the pipe system or supplying the fermentation can be provided. In this way, layers of the algae can then be prepared for the photobioreactor or for a biogas unit.

Preferably, the pipes on one level are disposed in a spiral shape. The pipes can be configured as a double spiral, in such a way that each double spiral has several windings, e.g. 30 windings. In order to guide the daylight to the pipes in an improved manner, the pipes are preferably disposed in a vertically offset manner. In a preferred embodiment, pipe sections of the levels can be separated from each other in order to enable an improved maintenance.

FIG. 2 illustrates the structure of a level with a retaining mechanism 15 for the transparent pipe system. The retaining mechanism 15 preferably comprises several retaining members 16 which, in particular, have guides 17 for accommodating the pipes. The guides 17 can be disposed vertically offset on the retaining members so that an optimized lighting of adjacent pipes is ensured. In the case of FIG. 2, the retaining members 16 are disposed in a spoke shape about an internal frame 18 in order to occupy the substantially cylindrical space in a space-saving manner. The internal frame 18 can be adjacent to an optional fermenter 11. On the outside, the retaining members 16 can be attached to a corresponding external frame 19. For this purpose, the retaining member 16 can be inserted into U-profiles and glued thereto, for example. In particular, the retaining members 16 are in this case uniformly distributed about the circumference in order to enable a support of the transparent pipe system on the level that is equal to the load. For example, the retaining members 16 are positioned at an angle of 3°, respectively, so that the supporting distance 21 on the outside is, for example, 115 cm. Preferably, the maximum supporting distance of the retaining members 16 is no more than 250 cm, particularly preferably no more than 150 cm.

The retaining members 16 preferably consist of plastic, such as rigid foam or polystyrene, with the underside of the retaining members 16 being fabricated, in particular, from glass fiber-reinforced plastic in order to ensure a sufficient strength in that area. Alternatively or additionally, a profile, in particular a U-shaped rail, is provided on the lower side of the retaining members 16, which contributes to the improved stability of the retaining mechanism 15 on the whole. The thickness of the retaining members 16 is preferably 20-80 mm, particularly preferably 40-60 mm.

The transparent pipe system is particularly advantageously arranged in two vertically and laterally offset tube spirals. Preferably, the guides 17 of the respective retaining members 16 are disposed slightly offset as compared with adjacent retaining members. The offset of the guides 17 from the respectively adjacent retaining member is, for example, between 15 and 20 mm, so that a spiral-shaped arrangement is thus produced. The arrangement of the adjacent pipes is configured in such a way, in particular, that the centers of the pipes form an equilateral triangle.

Light-transmissive supporting members, such as peripheral intermediate support rings 6, preferably comprise a profiled rail, for example a U-shaped support 13, and a framework structure by means of connecting tubes 14. A post or skeleton structure can basically also be provided. In particular, the supporting members are made from steel. The U-shaped supports 13 and the connecting tubes 14 are in this case preferably welded to each other as a composite structure. The lower U-shaped support 13 can then be placed on an edge of a concrete member, such as a concrete ring 12, for example. In the same way, another concrete ring 12 can be placed on the upper U-shaped support 13. The assembly expenditure can consequently be reduced. The edge of the concrete members can also be rounded for an improved placement of the U-shaped supports 13.

FIG. 3 illustrates the movable embodiment of a level. For this purpose, the external frame 19 can have rollers 20, particularly on the circumference of the external frame. In a preferred embodiment, a corresponding track 24 for the rollers 20 is provided on the intermediate support ring 6, preferably as an appendage of the lower U-shaped support 13, and thus enables a horizontal movement, i.e. a rotation of the level. In addition, supporting rollers 25 can also be provided on the external frame 19, which are supported in particular on a vertical side of the intermediate support ring 6 in order to enable a secure guidance of the level during rotation. The pipes or pipe sections of one level can thus be handled in an improved manner for maintenance. Correspondingly, the internal frame 18 comprises roller and/or supporting rollers rolling on corresponding tracks. The number of rollers on the external frame 19 for one level is, for example, 6-18. The number of rollers on the internal frame 18 typically is lower than in the case of the external frame 19.

In a preferred embodiment, the external frame 19 further has a means for vertically moving the levels. For this purpose, one or more driven gears 23 can be provided on the external frame 19, which mesh with a corresponding toothed rack 22 in order to enable a vertical movement of the level. The number of rack-and-pinion drives on the external frame 19 for one level is, for example, 6-18. In that case, fewer rack-and-pinion drives, for example half as many, can be provided on the internal frame 18.

In a preferred embodiment, the levels can be moved to the height of the light-transmissive supporting members, i.e. the intermediate support rings 6, for example. In particular, the intermediate support rings 6 are then equipped with tracks for rotating the levels. The structural design for making the transparent pipe system accessible for maintenance can thus be simplified. In that case, a level is first vertically moved to the closest intermediate support ring 6. Then, the level can be rotated in such a way that a desired pipe or pipe section is accessible for maintenance. In another embodiment, an extension arm can be provided, which enables an improved handling when laying or removing the pipes. It is particularly advantageous to provide at least two intermediate support rings 6 in the wall region 2 of the outer shell. This structure contributes to the levels having to be moved less in order to be accessible from the intermediate support rings 6. Since the levels preferably have a minimum spacing from one another, the required structural height as a whole can thus be reduced. In the case of a division with two intermediate support rings 6, the structural height can typically be reduced by half. For example, the individual levels have a minimum spacing of at least 0.4 m. The height of the intermediate support rings 6 typically is no more than 100 cm, more preferably no more than 70 cm.

The track 24 of, in particular, an intermediate support ring 6 is preferably configured in such a way that it can be opened in some sections. The track can be interrupted, for example, by swinging open or extending a track segment. The protruding rollers 20 on the external or internal frame can in this way pass the tracks during vertical movement. Subsequent to vertical movement, the track can be closed so that the level can be placed on the closed track by means of the rollers 20. In order to enable the rotation of the levels, the toothed racks 22 can be taken out of engagement with the gears 23. The weight of a level, with filled pipes, is between 5 and 30 t, for example. In particular, it is advantageous to provide a machine-powered rotating device and/or lifting device for the levels.

FIG. 4 schematically illustrates the arrangement of the pipes 26 on the retaining mechanism 15 of the pipe system. As is apparent from FIG. 4, the pipes 26 are arranged in such a way that the centers of the respective pipes 26 form an isoceles triangle. The pipes 26 are accommodated in vertically offset guides of the retaining member 16. In particular, a lighting element 27, for example with LED lamps or the like, is disposed between the vertically offset pipes 26. Here, the lighting element 27 lies on the lower pipe 26 and thus in the region that is shaded to a particular extent. Accordingly, this shaded region can be illuminated particularly optimally.

A particularly stable cohesion between the pipe system and the retaining mechanism 15 can be achieved by the pipes 26 being connected to the retaining member 16 in a positive and/or frictional manner at least in some areas. A fastening member which firmly connects the pipes 26 to the retaining mechanism 15 can be provided for this purpose. In the case of FIG. 4, a clip 26 is provided which is tightened about the pipes and fixated on the retaining member 16 by means of eyelets 29. In principle, it is also possible to provide a wire or the like as a fastening member. Preferably, the contact area between the pipe 26 and the retaining member also comprises a locking means in order to obtain a firm connection between the pipe system and the retaining member 15. A disengageable locking means is preferred in that it also ensures a flexible maintenance of the pipe system. Here, a hook-and-loop fastener 31 is provided as a disengageable locking means in the contact area, i.e. substantially in the guides 17 of the retaining mechanism 15. The cohesion between the pipe system and the retaining mechanism is thus improved, so that a load carrying capacity is obtained that is, on the whole, improved.

It is also constructionally advantageous to provide a reinforcing member 28 at the lower portion of the retaining member 16. The reinforcing member 28 preferably consists of a more solid material than the rest of the retaining member 16. Thus, the retaining member 16 can in principle be fabricated from a light plastic, such as polystyrene, with the reinforcing member 28 consisting of a glass fiber-reinforced plastic or graphene. It is also advantageous to configure the reinforcing member 28 in the form of a rail, particularly with a U-shaped profile, in order to make a sufficient stability and strength of the retaining mechanism 15 possible at a low weight.

FIG. 5 schematically shows a basement 32 of the photobioreactor or of the biogas unit, which is configured, in particular, as a basement level. The basement can be divided into different segments in order to provide different functions of the photobioreactor or the biogas unit. In the case of a biogas unit, it is particularly advantageous to dispose all further unit components, such as the storage areas, tanks, pumps, supplying equipment and/or harvesting device underneath the photobioreactor, in particular in a basement (32). This makes a particularly space-saving arrangement of the unit components possible. Furthermore, this makes a closed unit possible that, apart from the reduced space requirements, also differs from the prior art described in the introduction particularly in that the environmental impact, such as air pollution or noise, is contained. This also increases the fire and traffic safety of a photobioreactor or a biogas unit. In addition, the expenditure that the transport of biosubstrates entails is reduced considerably, because the biowaste masses of the local area can be used expediently.

In order to reduce the danger of fire, the photobioreactor or the biogas unit are preferably equipped with a fire extinguishing system. The latter is preferably configured in such a way that the water conducted in the photobioreactor is at least partially used as an extinguishing agent. Accordingly, at least one output device, such as a valve, a nozzle, a sprinkler or the like can be provided on the pipe system. Particularly preferably, an output device is provided on each level of the pipe system. In the case of a fire hazard, the liquid conducted in the pipe system of the photobioreactor, such as water, for example, can be partially released in large quantities. It is also possible to release stored nitrogen, e.g. from nitrogen bottles, in the closed system in order thus to cause the flames to be extinguished. It is particularly advantageous to configure the fire extinguishing system for automatic operation.

A machine compartment 44 for the fermenter 11 is preferably provided in the center of the basement 32. In particular, it includes agitators 34 for an agitation in the fermenter 11. The agitators 34 are driven, in particular, by electric motors. For a particularly space-saving arrangement, the agitators 34 or electric motors can be provided on the ceiling of the basement.

Furthermore, the basement 32 can include storage areas 33, for example for algae, tanks 37, for example for slurry, machine compartments 38, for example for pumps, and staff rooms 39. Preferably, filling devices, such as bulk material devices 41, are provided at the basement. They enable a fast delivery from the outside, particularly using trucks 42. Access paths 35 ensure the internal access in the basement 32.

In order to enable a transport of the stored material from the storage areas 33 or the tanks 37 for a supply into the fermenter 11, conveying pipes 36 or conveyor devices 43, such as conveyor screws, can be provided, depending on the material to be conveyed.

Preferably, the photobioreactor comprises at least one bypass or bypass port for the removal of cultivated phototrophic organisms, such as algae. Preferably, a bypass or bypass port is provided on every level of the pipe system. The complete removal of the phototrophic organisms is then carried out, in particular, in the basement. Preferably, a harvesting device is provided for removal, which is configured in such a way that cultivated phototrophic organisms, such as algae, can be removed and divided into substrates. The divided young algae are then separately supplied to the pipe system for a growth circulation. Since the pipe system, in particular in the form of a water system, is preferably configured in a closed manner in order to keep the required pumping energy low, the arrangement of the harvesting device in the pipe system is such that a bypass can be opened segment by segment. Thus, cultivated algae can be supplied to the bypass in order to harvest them by means of the harvesting device. The harvesting device is advantageously provided in the basement, for example in a preparation room 40.

In order to provide as optimal conditions as possible for the cultivation of the phototrophic organisms, it is particularly advantageous to provide a two-stage or multi-stage fermenter for the biogas unit. The various stages of the fermenter can then be disposed one atop the other or side-by-side in a particularly compact manner.

In another embodiment of the invention, the transparent pipe system comprises a feed and/or drain pipe, which is preferably integrated in the pipe system in a load-bearing manner. Preferably, the feed and/or drain pipe has a cross section of 3 cm to at least 6 cm. As a feed pipe, the pipe is, in particular, configured in such a way that treated water, for example enriched with enzymes or nutrients, can be fed into the transparent pipe system. This correspondingly improves the growth of the phototrophic organisms. As a drain pipe, the pipe is, in particular, configured in such a way that water that is not required, for example wastewater or replaced water, can be discharged from the transparent pipe system. Accordingly, the pipe system is prevented from becoming dirty or congested.

The arrangement of the feed and/or drain pipes is preferably such that it is provided centrally in the pipes of the pipe system, which are substantially arranged in a triangular shape relative to one another. The triangular arrangement of the pipes is apparent from FIG. 4, wherein the feed and/or drain pipe can be provided alternatively or additionally to the lighting pipe 27. In order to make an improved load-bearing function of the pipe system possible, the feed and/or drain pipes, particularly advantageously, are firmly connected to the pipe system and thus forms a stable assembly. For this purpose—as was already described—fasteners such as clips or clamps of plastic or sheet metal can be provided, which are optionally supplemented with wire clamps or hook-and-loop fasteners. The feed and/or drain pipe consists, in particular, of a solid plastic, such as PVC, aluminum or steel. In another embodiment, a state monitoring system of the pipe system is provided which is capable of monitoring the state of the pipes in the individual levels. It is thus ensured that the, in particular, circulation-like pipe system is not congested by excessive dirt or that the growth of the phototrophic organisms is otherwise affected.

It is particularly advantageous to use the feed and/or drain pipe as a lighting pipe 27 at the same time. For this purpose, lighting means, such as light-emitting diodes (LED), which are particularly preferably configured as a coating in order to cause an emission of light over the entire surface, can be provided on the feed and/or drain pipe. This makes it possible to brighten up shading on the pipe system of the photobioreactor.

Furthermore, the pipe diameters serve for water management, as do possibly existing treatment basins in the basement, pumps and inlet and outlet valves on the pipe system of the photobioreactor, which is laid in a spiral shape. They are preferably configured in such a way that an automatic monitoring and regulation is made possible.

### REFERENCE NUMERALS

1 Photobioreactor
2 Wall region
3 Roof
4 Support structure
5 Roof cover
6 Intermediate support ring
7 Height of 2
8 Diameter of 1
9 Height of 3
10 Stair tower
11 Fermenter
12 Concrete ring
13 U-shaped support
14 Connecting tube
15 Retaining mechanism
16 Retaining member
17 Guide
18 Internal frame
19 External frame
20 Roller
21 Supporting distance
22 Toothed rack
23 Gear
24 Track
25 Supporting roller
26 Pipe
27 Lighting pipe
28 Reinforcing member
29 Eyelet
30 Clip
31 Hook-and-loop fastener
32 Basement
33 Storage area
34 Agitator
35 Access path
36 Conveying pipes
37 Tank
38 Machine compartment
39 Staff room
40 Preparation room
41 Bulk material device
42 Freight vehicle
43 Conveyor device
44 Machine compartment for 11

1. A photobioreactor for the cultivation of phototrophic organisms, in particular algae, comprising a transparent pipe system for the flow-through of a culture suspension, in particular algae substrate, wherein the transparent pipe system is configured in the form of levels.

2. The photobioreactor according to claim 1, comprising an outer shell that is light-transmissive in some regions, preferably comprising a light-transmissive roof (3).

3. The photobioreactor according to claim 1, wherein the roof is configured for a varying light transmittance and preferably has a double-walled structure for the flow-through of a gas or a magnetizable liquid, in particular with metal particles, wherein the double-walled structure further preferably has a graphene layer, preferably in such a way that the light transmittance of the graphene layer can be altered by the magnetizable liquid.

4. The photobioreactor according to claim 1, wherein the outer shell, in particular in the wall region, has at least one light-transmissive supporting member, and/or that the outer shell, in particular in the wall region, has one or more concrete members.

5. The photobioreactor according to claim 1, wherein the transparent pipe system is configured in a closed manner so as to provide a circulation process and that preferably the length of the pipe system extends over at least several kilometers.

6. The photobioreactor according to claim 1, wherein the transparent pipe system has separable sections, and/or the inner surfaces of the pipe system are coated with graphene.

7. The photobioreactor according to claim 1, wherein at least one level of the transparent pipe system comprises a retaining mechanism for the pipe or pipes and that preferably, the retaining mechanism is substantially configured in a spoke-shaped manner.

8. The photobioreactor according to claim 7, wherein the retaining mechanism is configured for a vertically and/or laterally offset arrangement of the pipes and that, preferably, a reinforcing member is provided in the lower region.

9. The photobioreactor according to claim 7, wherein the pipes of the transparent pipe system, at least in some regions, are connectable or connected to the retaining mechanism in a positive and/or frictional manner, preferably with a disengageable locking means.

10. The photobioreactor according to claim 1, wherein at least one level of the pipe system or a retaining mechanism for the pipes is configured to be horizontally and/or vertically movable and that preferably, means for horizontal and/or vertical movement are disposed on the roof.

11. The photobioreactor according to claim 1, comprising at least one vertical access element for access to the interior, in particular to the levels of the pipe system.

12. The photobioreactor according to claim 1, wherein the transparent pipe system comprises a feed and/or drain pipe, which is preferably integrated in the pipe system in a load-bearing manner.

13. An arrangement of a light-transmissive supporting member for a, in particular in a photobioreactor for the cultivation of phototrophic organisms, in particular algae, wherein the supporting member is retained in the wall region of the outer shell of the photobioreactor, wherein the supporting member is configured in a closed manner in the horizontal extent and preferably has a framework structure.

14. The arrangement according to claim 13, wherein the height of the supporting member is no more than 100 cm, more preferably no more than 70 cm.

15. The arrangement according to claim 1, wherein the supporting member comprises profiled rails, in particular at the lower and/or upper end, for connection to the further wall region of the outer shell of the photobioreactor.

16. A biogas unit for the production of biogas with a photobioreactor according to claim 1 and a fermenter preferably disposed in the interior of the photobioreactor.

17. The biogas unit according to claim 16, wherein the photobioreactor has a temperature regulating device that is capable of utilizing the heat of the fermenter, and/or that the fermenter is configured in a two-stage or multi-stage manner, preferably with stages located side-by-side or one atop the other.

18. The biogas unit according to claim 1, wherein all unit components, such as the storage area, tank, pump, supplying equipment and/or the harvesting device are disposed underneath the photobioreactor, in particular in a basement.

19. The biogas unit according to claim 1, wherein a fire extinguishing system is provided, which is connected, in particular in a liquid-conducting manner, to the closed circulation of the pipe system, and is preferably configured in such a way that the liquid conducted in the pipe system can be used as an extinguishing agent, wherein, further preferably, at least one output device, in particular a valve, a nozzle, a sprinkler or the like is provided for every level of the pipe system.

20. The biogas unit according to claim 1, wherein the pipe system comprises at least one input device, in particular a valve or the like, for supplying carbon dioxide or crude gas, in particular biomethane, preferably in such a way that the input device is disposed in every level of the pipe system.

21. (canceled)
